(19) United States
(12) Patent Application Publication
(10) Pub. No.: US 2022/0087662 A1
(43) Pub. Date: Mar. 24, 2022

(54) MEDICAL SYSTEM HAVING DETACHABLE END EFFECTOR WITH ARTICULATION

(71) Applicant: Boston Scientific Scimed, Inc., Maple Grove, MN (US)

(72) Inventor: Joseph W. KING, Franklin, MA (US)

(73) Assignee: Boston Scientific Scimed, Inc., Maple Grove, MN (US)

(21) Appl. No.: 17/477,942

(22) Filed: Sep. 17, 2021

Related U.S. Application Data
(60) Provisional application No. 63/081,670, filed on Sep. 22, 2020.

(51) Int. Cl. A61B 17/00

(52) U.S. Cl. CPC A61B 17/00; A61B 2017/00323; A61B 2017/00367; A61B 2017/00473

(57) ABSTRACT

A medical device includes a handle having a proximal end, a distal end, and an actuator to move between the distal end and the proximal end, where the actuator is connected to a handle actuation wire, a tube extending from the distal end of the handle, and a connector at a distal end of the tube, the connector is detachably connected to an end effector. A distal end of the handle actuation wire includes a connector element to detachably connect to a corresponding connector element of an end effector actuation wire of the end effector.

### CROSS-REFERENCE TO RELATED APPLICATIONS

This patent application claims the benefit of priority under 35 U.S.C. § 119 to U.S. Provisional Patent Application No. 63/081,670, filed Sep. 22, 2020, which is herein incorporated by reference in its entirety.

### TECHNICAL FIELD

This disclosure relates generally to medical devices and methods of use. More particularly, in some embodiments, the disclosure relates to tools, systems, and/or methods for performing a medical procedure at a target site in a patient for, e.g., fixing or fastening tissue or performing any other diagnostic or therapeutic procedure, using a selectable end effector with articulation.

### BACKGROUND

Conventional endoscope devices for performing medical procedures in a patient are sometimes provided as single-use devices. These single-use devices are intended to perform a single procedure, e.g., clip or cut tissue. These single-use devices may also be dependent on articulation of an independent catheter or scope to articulate an end effector of the single-use device. Sterilization of these endoscope devices can be difficult, and failure to properly sterilize these devices can be harmful to patients by, e.g., spreading disease. Moreover, multiple uses of these single-use devices may increase a patient risk of disease or infection and/or may increase a risk of device failure. Further, the inability to articulate the end effector of the device, independent of the catheter or scope, reduces its mobility.

This disclosure may solve one or more of these problems or other problems in the art. The scope of the disclosure, however, is defined by the attached claims and not the ability to solve a specific problem.

### SUMMARY OF THE DISCLOSURE

According to an aspect, a medical device includes a handle having a proximal end, a distal end, and an actuator configured to move between the distal end and the proximal end, wherein the actuator is connected to a handle actuation wire, a tube extending from the distal end of the handle, and a connector at a distal end of the tube, wherein the connector is configured to detachably connect to an end effector, wherein a distal end of the handle actuation wire includes a connector element configured to detachably connect to a corresponding connector element of an end effector actuation wire of the end effector.

The medical device may further comprise the end effector, and wherein the end effector may be a medical tool, and movement of the actuator in at least one of the proximal direction and the distal direction may be configured to cause the medical tool to be actuated.

The actuator may be a spool, and the handle may include a handle body on which the spool translates.

The handle may further include at least one articulation control device, and wherein the at least one articulation control device may be connected to a handle articulation wire.

The at least one articulation control device is configured to move between the proximal end and the distal end of the handle, and wherein a proximalmost position of the at least one articulation control device may be distal of a distalmost position of the actuator.

The end effector may include an end effector articulation wire, wherein the handle articulation wire may be configured to engage the end effector articulation wire when the end effector may be connected to the connector, and wherein movement of the at least one articulation control device may be configured to move the end effector from a first position, wherein the end effector may be aligned with a longitudinal axis, to a second position, wherein at least a portion of the end effector may be angled relative to the longitudinal axis.

At least one articulation control device may include a plurality of articulation control devices, and wherein the plurality of articulation control devices may be radially spaced about the handle.

Each of the plurality of articulation control devices may be attached to a corresponding handle articulation wire.

The connector may include a distal facing cavity, and wherein a proximal portion of the end effector may be configured to be inserted at least partially into the cavity when the end effector is connected to the connector.

The end effector may include an extender attached at a proximal end to an end effector connector and at a distal end to a medical tool, wherein movement of the at least one articulation control device may bend the extender.

The end effector may include at least one end effector articulation wire disposed radially outward of the extender.

A distal end of the handle actuator wire may include a hook, wherein a proximal end of the end effector actuation wire may include a hook for connection to the hook of the handle actuation wire.

A proximal force applied to the handle may be configured to disconnect the end effector from the connector, and wherein the proximal force may be sufficient to deform at least one of the handle actuation wire and the end effector actuation wire to disconnect the end effector from the connector.

The connector may be configured to be attached to another end effector after the end effector is disconnected from the connector.

The medical device may further comprise a rotation control device connected to the handle and the tube, wherein rotation of the rotation control device may be configured to rotate the end effector about a longitudinal axis of the tube.

According to another aspect, a medical device includes a handle having a distal end, a proximal end, and an actuator configured to be moved between the distal and proximal ends, a tube extending from the distal end of the handle,

an actuation wire attached to the actuator and extending through the tube, wherein the actuation wire is configured to be removably connected to an end effector at a distal end of the tube, to actuate the end effector, at least one articulation device on the handle, wherein the at least one articulation device is configured to be moved between the distal and proximal ends, and at least one articulation wire attached to the articulation device and extending through the tube, wherein the articulation wire is configured to be removably connected to the end effector, to deflect the end effector.

The at least one articulation wire may be configured to connect to a corresponding articulation wire on the end effector, and wherein articulation of the at least one articulation device may be configured to cause the end effector to move from a first position, wherein the end effector is aligned with a longitudinal axis, to a second position, wherein at least a portion of the end effector is angled relative to the longitudinal axis.

A proximal force on the handle may be configured to separate the connector from the end effector.

According to another aspect, an end effector for use with a medical device may include a connector, an extender extending distally from the connector, a medical tool attached to the extender, an end effector actuation wire extending proximally from the medical tool and configured to connect to a corresponding actuation wire of the medical device, and at least one end effector articulation wire configured to connect to a corresponding articulation wire of the medical device, wherein movement of the at least one end effector articulation wire in at least one of a proximal direction or a distal direction is configured to bend the extender.

The end effector actuation wire may extend through a lumen of the extender, and wherein the at least one end effector articulation wire may be positioned radially outward of the extender.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a side view of a medical device according to an exemplary embodiment;

FIG. 2 is a cross-section of a portion of a catheter and a connector of the medical device of FIG. 1;

FIG. 3 is a perspective view of an end effector of the medical device of FIG. 1; and

FIG. 4 is a cross-section of an end effector connected to a connector of the medical device of FIG. 1.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

For ease of description, portions of the device and/or its components are referred to as proximal and distal portions. It should be noted that the term “proximal” is intended to refer to portions closer to a user of the device, and the term “distal” is used herein to refer to portions further away from the user. Similarly, extends “distally” indicates that a component extends in a distal direction, and extends “proximally” indicates that a component extends in a proximal direction. Further, as used herein, the terms “about,” “approximately” and “substantially” indicate a range of values within +/−10% of a stated or implied value. Additionally, terms that indicate the geometric shape of a component/surface refer only to approximate shapes.

As described above, a medical system, or a portion thereof, may be used multiple times to decrease medical waste. The medical system may include a universal connector which may attach to a corresponding connector of each of a variety of end effectors such as, e.g., snares, hemostatic clips, forceps, needles, cutting knives, ablation devices, and any other tool useful in a medical procedure. In this manner, a single medical system, or a portion thereof, may be used multiple times, and with multiple end effectors.

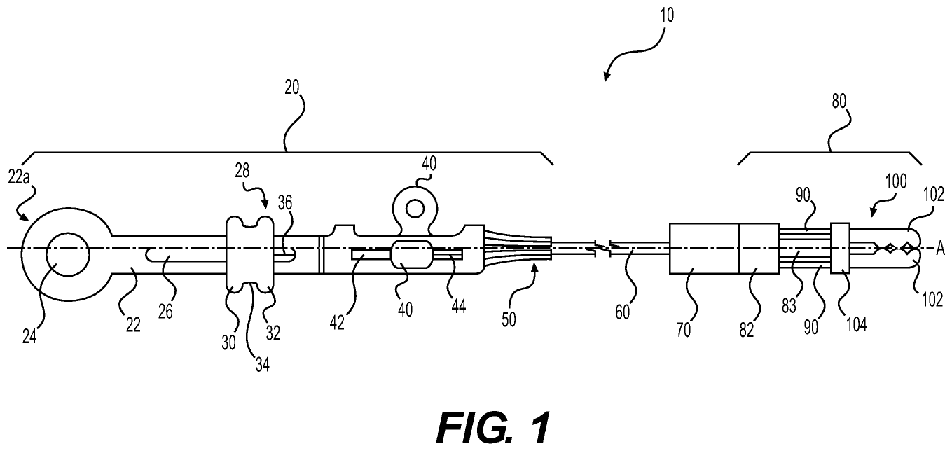

Referring to FIG. 1, a medical device 10 (e.g., a device for grasping tissue and/or dispensing medical clips) according to an embodiment is shown. Medical device 10 includes a handle 20, a catheter 60 (e.g., an outer sheath or tube defining a lumen) connected to a distal end of handle 20, a universal connector 70 connected to a distal end of catheter 60, and an end effector 80 (e.g., a clip for fastening tissue) connected to universal connector 70 via a connector 82.

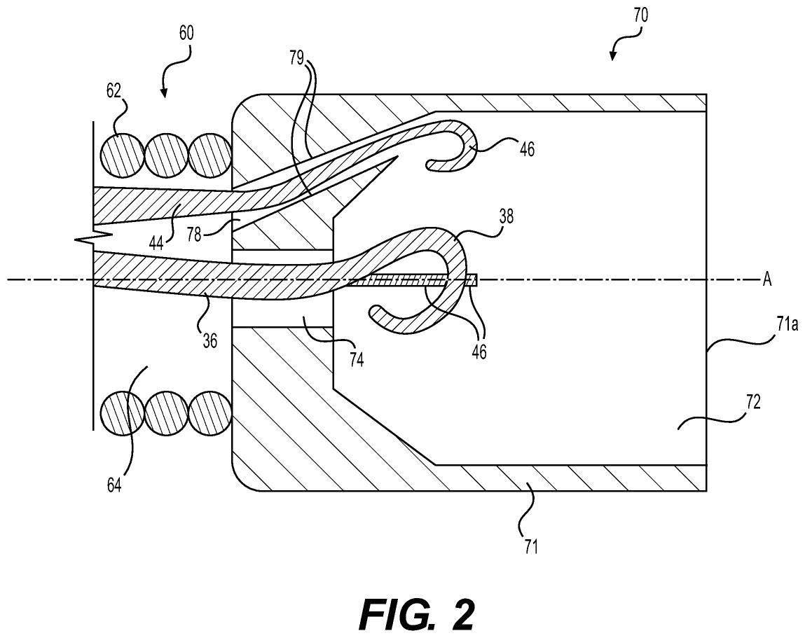

With continued reference to FIG. 1, handle 20 is illustrated. Handle 20 includes a body 22 defining a hole 24 in body 22 at a proximal end 22a thereof. Hole 24 can be used to accommodate a thumb of a user of medical device 10. Catheter 60 is attached at an opposite, distal end of body 22. A slot 26 extends through body 22 in a direction parallel to a longitudinal axis A of handle 20 and catheter 60. A spool 28 includes a bar (not shown) disposed in slot 26, and the bar moves within slot 26 and along body 22 in a direction parallel to longitudinal axis A. An actuation wire 36 (e.g., a wire or any other elongate actuator, such as a cable, a braided member, etc.) extends from the distal end of the bar distally through handle 20, catheter 60, and into cavity 72 of connector 70 (FIG. 2). As will be described herein, actuation of spool 28 in a proximal direction relative to body 22 causes wire 36 to actuate end effector 80. Spool 28 includes two annular protrusions 30, 32 at a distal end and a proximal end thereof, respectively, and extending from spool 28 in a direction perpendicular to the direction of longitudinal axis A. Annular protrusions 30, 32 define therebetween an annular grip 34, which is grasped by a user (e.g., by a middle finger and an index finger) as will be described in greater detail herein.

As further shown in FIG. 1, a slot 42 is defined in a sidewall of body 22, distal of slot 26 and spool 28. Slot 42 extends from a position distal of a distalmost end of slot 26 and extends in a distal direction, parallel to longitudinal axis A. While only one slot 42 is shown, multiple slots 42 may be arranged axially about body 22 of handle 20, e.g., such that slots 42 are evenly spaced about body 22. For example, device 10 includes two slots, one slot 42 shown in FIG. 1, and the other slot 42 in the top of body 22 in FIG. 1. A knob 40 may slide within each slot 42 in a direction parallel to longitudinal axis A. Knob 40 may include a hole in which a user may insert a finger or a thumb to assist in movement of knob 40. Alternatively, or additionally, each knob 40 may be shaped such that a user may grasp knob 40, e.g., with a finger and/or a thumb, to slide each knob 40 proximally and distally within corresponding slot 42.

A distal end of each knob 40 may be attached to a proximal end of a corresponding articulation wire 44 (e.g., a wire or any other elongate actuator, such as a cable, a braided member, etc.). Wire 44 extends from knob 40 in a distal direction, through body 22 of handle 20 and catheter 60, terminating in cavity 72 of connector 70 (FIG. 2). As will be explained herein, movement of knob 40 and corresponding wire 44 in a proximal-distal direction may articulate a distal end of medical device 10 (e.g., the end effector, medical tool, and/or other portions of the distal end of medical device 10) right, left, up, or down relative to longitudinal axis A. For example, each knob 40 may be attached to a corresponding wire 44. Medical device 10 may include one, two, three, or four (or more) sets of knobs 40 and wires 44. Actuation of knobs 40 and corresponding wires 44 may provide actuation of medical device 10 independent of an access catheter or scope. In this manner, a distal end of medical device 10 (e.g., including end effector 80) may be articulated distal of a distalmost end of the access catheter or scope.

With continued reference to FIG. 1, catheter 60 (e.g., a wire coil, a sheath, or the like) may be attached to the distal end of handle 20 via a rotation control device 50. Rotation control device 50 may include ridges, protrusions, a friction-increasing coating, or an otherwise non-uniform surface to improve the gripping surface for a user's fingers. During use, the user may use grasp rotation control device 50, e.g., using a thumb and a forefinger, to rotate rotation control device 50 about longitudinal axis A. Rotation of rotation control device 50 may rotate catheter 60 and connector 70, which may also rotate end effector 80 connected to connector 70. In this manner, the user may align end effector 80 with a target site about longitudinal axis A with a target tissue to perform the medical procedure.

With reference to FIG. 2, a cross-section of the distal end of catheter 60 is shown connected to a proximal end of connector 70. Catheter 60 includes a central lumen 64 defined by an outer wall 62. Outer wall 62 may be a wire coil or other suitable material for use in a medical device to enable a rotation about longitudinal axis A based on a rotation of rotation control device 50. Outer wall 62 may be attached to connector 70 via welding, an adhesive, or other suitable attaching mechanism.

Connector 70 includes a body 71 defining cavity 72. Body 71 may be any medical grade material suitable for medical procedures. Body 71 may define a distal-facing opening 71a and which may receive connector 82, as will be described herein. At a proximal end, connector 70 may include a central opening 74. Wire 36 may extend from handle 20, through lumen 64, and into cavity 72 through central opening 74. A distal end of wire 36 may include a hook 38, which may connect to a corresponding site on end effector 80. Wire 36 may be formed of a flexible material suitable to bend, such that hook 38 may bend from the hook shape to an elongated shape, as will be described herein.

Connector 70 may also include one or more articulation openings 78 extending through the proximal end of connector 70 (for ease of understanding, a single articulation opening is shown in FIG. 2). Articulation openings 78 may be arranged radially about central opening 74. According to an example, each articulation opening 78 may be angled radially outward from a proximal end of each articulation opening 78 toward a distal end thereof. For example, walls 79 may define articulation opening 78 and may be angled from the proximal end of articulation opening 78 to the distal end of articulation opening 78. Articulation wire 44 may pass from handle 20, through lumen 64, and into cavity 72 via articulation opening 78. Articulation openings 78 may guide corresponding articulation wires 44 as articulation wires 44 are actuated. Each articulation wire 44 may include a hook 46 or other connection mechanism at a distal end thereof to connect to a corresponding element in end effector 80, as will be described herein. Each articulation wire 44 may be formed of a flexible material suitable for bending when a sufficient force is applied thereto, such that hook 46 may move from the hook shape to an elongate shape, as will be described herein. As articulation wires 44 are moved in a proximal and distal direction, end effector 80 may be moved left, right, up, and/or down relative to longitudinal axis A. In FIG. 2, an articulation opening 78 (not shown) is behind central opening 74.

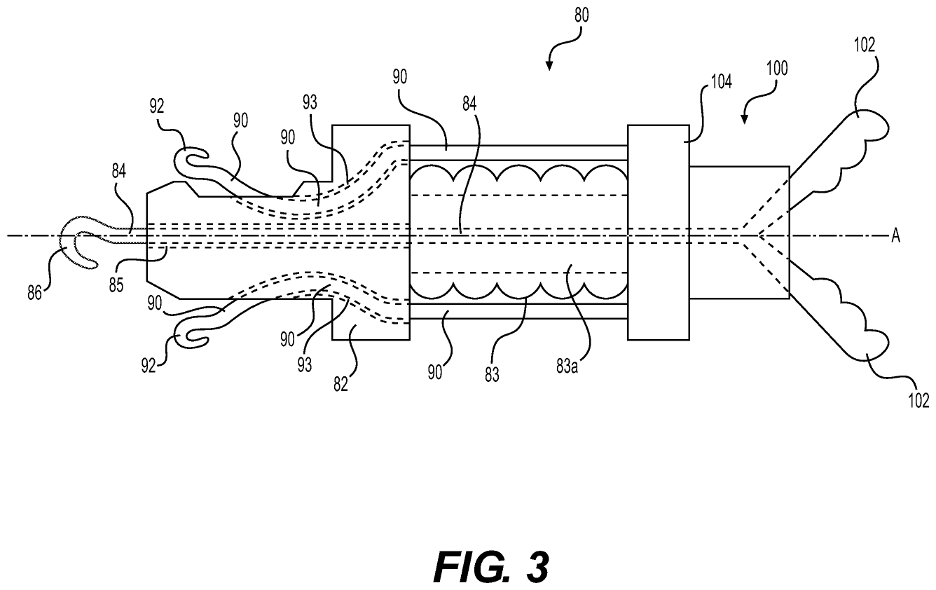

With reference to FIGS. 1 and 3, an end effector 80 is shown with a universal connector 82 configured to mate with connector 70. End effector 80 is shown connected to connector 70 in FIG. 1. End effector 80 may include an extender 83 extending from a distal end of connector 82. Extender 83 may include a lumen 83a through which one or more wires or cables may extend. A distal end of extender 83 may be attached to a proximal end of a medical tool 100. Extender 83 may be a spring, a coil, a bellows, or the like, which is configured to bend in one or more directions. According to an example, medical tool 100 may be a grasper, having a base 104 attached to the distal end of extender 83, and a pair of opposing forceps 102. As will be explained herein, actuation of wire 44 may actuate forceps 102 to cause them to open and close. In this manner, a user may grasp tissue or other material between forceps 102. Medical tool 100 is not limited to the grasper, and may be any medical tool, such as but not limited to snares, biopsy forceps, needles, or the like.

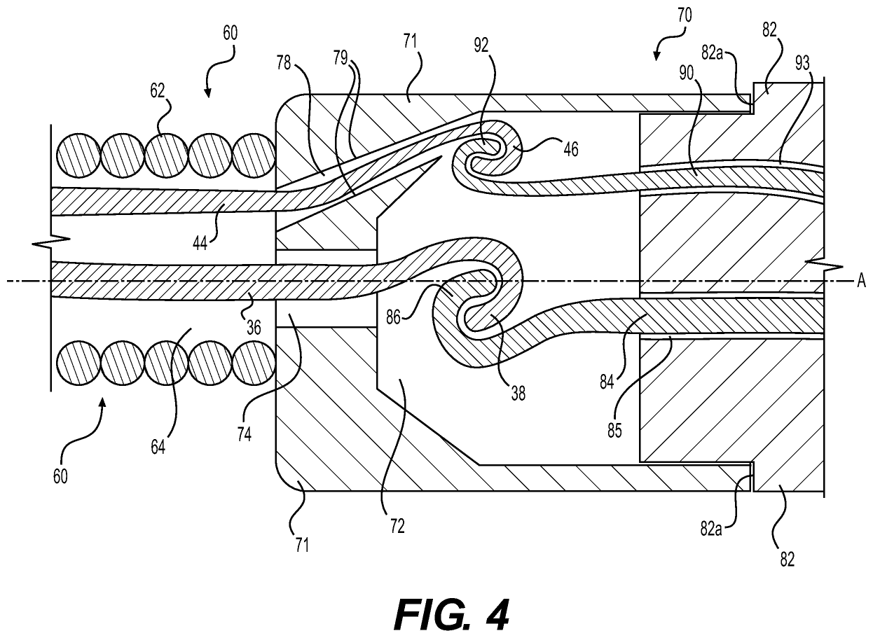

With reference to FIGS. 3 and 4, connector 82 includes a central opening 85 extending from a proximal end to a distal end thereof. Central opening 85 may be connected to be in fluid communication with lumen 83a of extender 83. An actuation wire 84 (e.g., a wire or any other elongate actuator, such as a cable, a braided member, etc.) may pass from the proximal end of connector 82, through central opening 85 and extender 88, and may be attached to forceps 102 via a clevis (not shown) or other suitable device for assembling forceps 102 to one another and to wire 84 to permit opening and closing of forceps 102, including any such device known in the art. A proximal end of wire 84 extends proximally of the proximal end of central opening 85 and forms a hook 86. Hook 86 may be any suitable connector for connecting to hook 38 of wire 44. Wire 84 may include a flexible material suitable for bending when a sufficient force is applied thereto, such that hook 86 may move from a hook shape to an elongate shape.

With continued reference to FIGS. 3 and 4, one or more openings 93 (only one opening 93 shown in FIG. 4 for ease of understanding) may extend from the proximal end of connector 82 to the distal end of connector 82. Alternatively, a proximal end of one or more openings 93 may extend through a radial outer surface of connector 82. Each opening 93 may have a straight path or a curved path. An articulation wire 90 (e.g., a wire or any other elongate actuator, such as a cable, a braided member, etc.) may extend through a corresponding opening 93 and may connect to base 104 at a distal end of articulation wire 92, via any suitable adhesive. A proximal end of each articulation wire 90 may include a hook 92 or other suitable connector. Articulation wire 90 may include a flexible material suitable for bending when a sufficient force is applied thereto, such that hook 92 may move from a hook shape to an elongate shape.

A connection between connectors 70 and 82 is shown in FIG. 4. An annular or circumferential flange 82a may be formed distal of a proximalmost end of connector 82, such that a portion of connector 82 proximal of flange 82a may have an outer diameter less than an inner diameter of cavity 72. When connectors 70 and 82 are connected by inserting the portion of connector 82 proximal of flange 82a into cavity 72, flange 82a may contact a distal facing end surface of connector 70. When connectors 70 and 82 are connected together, hooks 38 and 86 may clip (or “lock”) together, thereby connecting wires 44 and 84. In this manner, a movement of spool 28 may cause wires 44 and 84 to move in tandem, thereby actuating forceps 102 as will be described herein. Connectors 70 and 82 may have a shape and/or a mechanism or device (e.g., a protrusion on connector 70 and a corresponding receptacle on connector 82, or alignment markings on connectors 70, 82) to align connector 70 and 82 to assist in proper connection. Further, such structural shapes and/or mechanisms may assist a user to determine if end connector 82 is a suitable end connector for use with medical device 10.

Connecting connectors 70 and 82 together may also cause each hook 46 to clip (or “lock”) with a corresponding hook 92, thereby connecting each articulation wire 44 with a corresponding articulation wire 90 (for ease of understanding, a single actuation wire 44 and a single actuation wire 90 are shown in FIG. 4). In this manner, a movement or actuation of knobs 40 may cause corresponding actuation wires 44 and 90 to move in tandem, thereby causing end effector medical 100 to move right, left, up, and/or down. It will be understood that handle 20 may include more knobs 40 (and corresponding actuation wires 44) than actuation wires 90 contained in end effector 80. For example, handle 20 may include four knobs 40 corresponding to four actuation wires 44 to allow for actuation in four directions, e.g., up, down, left, and right. However, end effector 80 may be designed to move in less than four directions, e.g., only in a left/right direction, and may, thus, only include two actuation wires 90 on radially opposite sides of end effector 80. In this case, hooks 92 of each actuation wire 90 connect to hooks 46 of corresponding actuation wires 44, such that actuation of only two of the four actuation wires 44 will cause articulation of end effector 80. Thus, while handle 20 may provide more articulation directions than provided by an end effector, handle 20 may be used with end effectors having less articulation directions than that provided by handle 20.

As described herein, wires 36, 84 and articulation wires 44, 90 may include a flexible material that may bend when a sufficient force is applied thereto. For example, to disconnect connector 82 from connector 70, handle 20 may be moved in a proximal direction with a sufficient force to cause hooks 38, 86 and hooks 46, 92 to move from the hook configuration to an elongate configuration, thereby disconnecting corresponding wires. Alternatively, or additionally, a user may first move each knob 40 in a distal direction prior to moving handle 20 in the proximal direction. In this manner, tension between hooks 46, 92 may be reduced, thereby reducing the amount of force necessary to disconnect hooks 38, 86. Once hooks 38, 86 are disconnected, further proximal movement of handle 20 may disconnect hooks 46 from corresponding hooks 92.

A method of using medical device 10 will now be described. A user may first select an end effector from a variety of end effectors provided, for example, in a kit. Connector 82 of the selected end effector 80 may be attached to connector 70 by inserting the portion of connector 82 proximal to flange 82a into cavity 72. In this manner, hooks 38, 86 may connect. Further, one or more hooks 92 may connect to a corresponding hook 46. In this manner, end effector 80 may be connected or “locked” to connector 70.

After connecting end effector 80 to connector 70, end effector 80 may be inserted into a patient via an opening, e.g., an incision or a natural orifice, and advanced to a target site within the body. Alternatively, end effector 80 may be advanced to the target site via a catheter or scope that is pre-positioned in the body (for example, a catheter having working channel with a diameter approximately 3.5 mm to approximately 4.5 mm).

Once end effector 80 is positioned at the target site, a user may control end effector via handle 20. For example, the user may rotate end effector 80 by grasping and rotating rotation control device 50. In addition, the user may move end effector 80 relative to longitudinal axis A of medical device 10, e.g., up, down, right, or left, by moving one or more knobs 40 in a proximal or a distal direction along handle 20. For example, sliding the top knob 40 shown in FIG. 1 proximally may pull the top wires 44, 90 shown in FIG. 4 proximally, causing extender 83. Once end effector 80 is properly positioned relative to the target site, the user may actuate jaws 102 of medical tool 100 by moving spool 34 in the proximal and the distal direction to grasp tissue or another object at the target site. It will be understood that moving spool 34 may actuate a different medical tool, e.g., a snare or a knife, to perform a different medical procedure at the target site.

Once the medical procedure is performed, and in the event end effector 80 is designed to remain in at the target site (e.g., a hemostatic clip), connector 70 may be disconnected from connector 82. To disconnect connectors 70, 82, the user may grasp handle 20 and pull medical device 10 in the proximal direction with a force sufficient to bend and/or break hooks 38, 46, 86, and/or 92. For example, a material of hooks 86, 92 may be thinner or may have a greater flexibility than hooks 38, 46. In this case, hooks 86, 92 may be deformed when a sufficient force in the proximal direction is applied to handle 20. Once hooks 38, 46 disengage from corresponding hooks 86, 92, catheter 60 and connector 70 may be removed from the body. Alternatively, in the event end effector 80 is not designed to remain in the body, end effector 80 may be removed from the body along with catheter 60 and connector 70, and end effector 80 may be disconnected from connector 70 after removal from the body.

Once removed from the body, another end effector 80 selected from the variety of end effectors may be attached to connector 70. In one example, a same end effector 80 may be attached (e.g., a second hemostatic clip), or a different end effector 80 may be used, and the procedure may be performed in a similar manner discussed herein. In some instances, handle 20, catheter 60, and/or connector 70 may be discarded by the user, or may be sterilized and reprocessed or reused by the user using the same or a different end effector 80. Alternatively, handle 20, catheter 60, and connector 70 may be disinfected and returned to a manufacture to be reloaded and reused. In this manner, handle 20, catheter 60, and connector 70 may be reused in multiple procedures, which may decrease cost and/or decrease waste from medical procedures. In addition, since connector 70 may be used with multiple different medical tools 100, a single medical device 10 may be used to perform multiple different actions (e.g., cutting, grasping, and/or capturing a target tissue) during a medical procedure, which may save time, money, and/or other resources.

It will be understood that the portion of body 22 including slots 42 may be formed as a single piece as the portion of body 22 including slot 26, or separately from the portion of body 22 including slot 26. In one instance, the portion of body 22 including slots 42 may be formed as a separate member. In this case, wire 36 may be passed through a central lumen (not shown) of the portion of body 22 having slots 42 from a distal end to a proximal end, and attached to spool 28. The portion of body 22 having slots 42 may be attached in any manner to the portion of body 22 having slot 26, including but not limited to an adhesive, welding, a snap-fit, or the like.

Wires of handle 20 and end effector 80 are shown with hooks, but are not limited thereto. For example, corresponding wires may have a ball-and-socket connection, such that one wire has a spherical shaped end, and the corresponding wire has a correspondingly shaped receptacle. Alternatively, or additionally, some of the wires may include a material requiring a lower force to break than other wires. For example, wires of the end effector may break when a sufficient force is applied in the proximal direction on handle 20, while wires of handle 20 may not break. In this manner, it may be easier to release end effector 80 from handle 20 when end effector 80 is designed to remain in the body and/or when switching end effectors during a medical procedure. Alternatively, or magnets may be used to connect corresponding ends of wires. It will also be understood that disconnection of end effector 80 may be achieved without handle 20. For example, handle 20 may be configured so that it cannot provide sufficient force to disconnect end effector 80. Instead, disconnection may occur only when end effector 80 is outside of the body. Such disconnection may be done by a user simply pulling end effector 80 off of connector 70, by hand or by using a tool (e.g., pliers). In this manner, a user may not cause accidental disconnection of end effector 80.

It will also be understood that medical device 10 may be made of any material known in the art, including, but not limited to, a medical grade plastic or rubber, a ceramic, a metal, or a combination thereof.

It will be apparent to those skilled in the art that various modifications and variations can be made to the disclosed device without departing from the scope of the disclosure. For example, the medical device may be used to connect to various medial clips, knives, snares or other similar devices. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
a handle having a proximal end, a distal end, and an actuator configured to move between the distal end and the proximal end, wherein the actuator is connected to a handle actuation wire;
a tube extending from the distal end of the handle; and
a connector at a distal end of the tube, wherein the connector is configured to detachably connect to an end effector, wherein a distal end of the handle actuation wire includes a connector element configured to detachably connect to a corresponding connector element of an end effector actuation wire of the end effector.

2. The medical device according to claim 1, further comprising the end effector, and wherein the end effector includes a medical tool, and movement of the actuator in at least one of the proximal direction and the distal direction is configured to cause the medical tool to be actuated.

3. The medical device according to claim 1, wherein the actuator is a spool, and the handle includes a handle body on which the spool translates.

4. The medical device according to claim 1, wherein the handle further includes at least one articulation control device, and wherein the at least one articulation control device is connected to a handle articulation wire.

5. The medical device according to claim 4, wherein the at least one articulation control device is configured to move between the proximal end and the distal end of the handle, and wherein a proximalmost position of the at least one articulation control device is distal of a distalmost position of the actuator.

6. The medical device according to claim 4, wherein the end effector includes an end effector articulation wire, wherein the handle articulation wire is configured to engage the end effector articulation wire when the end effector is connected to the connector, and wherein movement of the at least one articulation control device is configured to move the end effector from a first position, wherein the end effector is aligned with a longitudinal axis, to a second position, wherein at least a portion of the end effector is angled relative to the longitudinal axis.

7. The medical device according to claim 6, wherein the at least one articulation control device includes a plurality of articulation control devices, and wherein the plurality of articulation control devices are radially spaced about the handle.

8. The medical device according to claim 7, wherein each of the plurality of articulation control devices is attached to a corresponding handle articulation wire.

9. The medical device according to 4, wherein the end effector includes an extender attached at a proximal end to an end effector connector and at a distal end to a medical tool, wherein movement of the at least one articulation control device bends the extender.

10. The medical device according to claim 1, wherein the connector includes a distal facing cavity, and wherein a proximal portion of the end effector is configured to be inserted at least partially into the cavity when the end effector is connected to the connector.

11. The medical device according to claim 10, wherein the end effector includes at least one end effector articulation wire disposed radially outward of the extender.

12. The medical device according to claim 1, wherein a distal end of the handle actuator wire includes a hook, wherein a proximal end of the end effector actuation wire includes a hook for connection to the hook of the handle actuation wire.

13. The medical device according to claim 1, wherein a proximal force applied to the handle is configured to disconnect the end effector from the connector, and wherein the proximal force is sufficient to deform at least one of the handle actuation wire and the end effector actuation wire to disconnect the end effector from the connector.

14. The medical device according to claim 13, wherein the connector is configured to be attached to another end effector after the end effector is disconnected from the connector.

15. The medical device according to claim 1, further comprising a rotation control device connected to the handle and the tube, wherein rotation of the rotation control device is configured to rotate the end effector about a longitudinal axis of the tube.

16. A medical device, comprising:
a handle having a distal end, a proximal end, and an actuator configured to be moved between the distal and proximal ends;
a tube extending from the distal end of the handle;
an actuation wire attached to the actuator and extending through the tube, wherein the actuation wire is configured to be removably connected to an end effector at a distal end of the tube, to actuate the end effector;
at least one articulation device on the handle, wherein the at least one articulation device is configured to be moved between the distal and proximal ends; and
at least one articulation wire attached to the articulation device and extending through the tube, wherein the articulation wire is configured to be removably connected to the end effector, to deflect the end effector.

17. The medical device according to claim 16, wherein the at least one articulation wire is configured to connect to a corresponding articulation wire on the end effector, and wherein articulation of the at least one articulation device is configured to cause the end effector to move from a first position, wherein the end effector is aligned with a longitudinal axis, to a second position, wherein at least a portion of the end effector is angled relative to the longitudinal axis.

18. The medical device according to claim 16, wherein a proximal force on the handle is configured to separate the connector from the end effector.

19. An end effector for use with a medical device, the end effector comprising:
a connector;
an extender extending distally from the connector;
a medical tool attached to the extender;
an end effector actuation wire extending proximally from the medical tool and configured to connect to a corresponding actuation wire of the medical device; and
at least one end effector articulation wire configured to connect to a corresponding articulation wire of the medical device, wherein movement of the at least one end effector articulation wire in at least one of a proximal direction or a distal direction is configured to bend the extender.

20. The end effector according to claim 19, wherein the end effector actuation wire extends through a lumen of the extender, and wherein the at least one end effector articulation wire is positioned radially outward of the extender.
